(19) United States
(12) Patent Application Publication
(10) Pub. No.: US 2007/0240241 A1
(43) Pub. Date: Oct. 11, 2007

(54) METHOD OF PREDICTING A TRAIT OF INTEREST

(75) Inventors: Maria Cristina Ubach, Chesterfield, MO (US); Luis A. Jurado, St. Louis, MO (US); Dutt V. Vinjamoori, Chesterfield, MO (US); Pradip Das, Olivette, MO (US); Bradley Krohn, Riverview, FL (US); Steven H. Modiano, Manchester, MO (US)

Correspondence Address: HARNESS, DICKEY & PIERCE, P.L.C., 7700 BONHOMME AVENUE, SUITE 400, ST. LOUIS, MO, 63105, US

(73) Assignee: Monsanto Technology LLC, St. Louis, MO (US)

(21) Appl. No.: 11/697,581

(22) Filed: Apr. 6, 2007

Related U.S. Application Data
(60) Provisional application No. 60/789,678, filed on Apr. 6, 2006.

(51) Int. Cl. A01H 1/00; G01N 1/30

(52) U.S. Cl. 800/284; 435/40.5

(57) ABSTRACT

A method of predicting a trait of interest from a plant by measuring the degree of starch-protein association in the plant is provided. The degree of starch-protein association can be determined by identifying either chemical properties or physical properties, or both chemical and physical properties of plant cells. In a particular embodiment, the degree of starch-protein association is determined by identifying the protein composition of the plant.

### CROSS-REFERENCE TO RELATED APPLICATION

This application claims priority to U.S. Provisional Application Ser. No. 60/789,678 filed Apr. 6, 2006. The disclosure of U.S. Provisional Application Ser. No. 60/789,678 is hereby incorporated herein by reference in its entirety.

### FIELD

The present invention relates to production of cereals and livestock feeds, and also relates to production of ethanol by fermentation of starch-containing plants. More specifically, the invention relates to a method of predicting a trait of interest, for example predicting high digestibility and/or predicting fermentability to produce high yield of ethanol.

### BACKGROUND

The statements in this section merely provide background information related to the present disclosure and may not constitute prior art.

Use of alternative energy sources can be desirable for several reasons, for example, reliance on fossil fuel may be decreased, and in turn air pollution may be reduced. Ethanol production by fermenting carbohydrate-containing plants is one possible source of alternative energy. For example, U.S. Pat. No. 4,568,644 to Wang et al. discusses a method for producing ethanol from biomass substrates by using a microorganism capable of converting hexose and pentose carbohydrates to ethanol, and to a lesser extent, acetic and lactic acids. U.S. Pat. No. 5,628,830 to Brink discusses a method for producing sugars and ethanol from biomass material which consists of two processes: hydrolysis of cellulose to glucose and fermentation of the glucose to ethanol.

Maximized ethanol production from biomass is economically desirable. Efforts have been made to achieve increased yield, especially by altering production processes or by adding extra steps for ethanol production. For example, U.S. Pat. No. 5,916,780 to Foody et al. discusses a process for improving economical ethanol yield by selecting feedstock with a ratio of arabinoxylan to total non-starch polysaccharides greater than about 0.39, then pretreating the feedstock to increase glucose production with less cellulose enzyme. Subsequent fermentation reportedly permits greater ethanol yield. U.S. Pat. No. 6,509,180 to Verser et al. discusses a process for producing ethanol including a combination of biochemical and synthetic conversions to achieve high yield ethanol production by preventing production of CO₂, a major limitation on the economical production of ethanol.

Maximized digestibility from biomass is also economically desirable. Grains grown and harvested for consumption by humans or by livestock have varying levels of digestibility. For livestock in particular, cost effective productivity and weight gain depends on the digestibility of the feed. The livestock feed industry has used several methods to improve feed value including steam flaking, reconstitution, micronisation, and high temperature, short-time extrusion. However, it would be more beneficial to predict prior to any processing step the digestibility of a particular plant variety, for example, the digestibility of a corn hybrid.

A number of techniques to characterize cellular organization of a plant are available. A plant's physical and/or chemical properties are used to analyze the plant's make-up. Chemical analysis is widely used in laboratories because it is fast and sensitive, and is suitable for automation.

Bietz, Separation of Cereal Proteins by Reversed-Phase High-Performance Liquid Chromatography, Journal of Chromatography, 255: 219-238 (1983) discusses the use of Reversed-Phase High-Performance Liquid Chromatography (RP-HPLC) to isolate, compare and characterize cereal proteins.

Bietz et al, in Wrigley (Ed.), Identification of Food-Grain Varieties, American Association of Cereal Chemists, St. Paul, 73-90 (1995) discuss the use of Reversed-Phase High-Performance Liquid Chromatography (RP-HPLC) to distinguish components of cereal plants, including maize, wheat, barley, rice, etc., as to genotype selection and identification in breeding.

Matrix-Assisted Laser Desorption Ionization Time-Of-Flight Mass Spectrometry (MALDI-TOF MS) can also be used for plant cell analysis. MALDI-TOF MS, due to its ease of use and relative insensitivity to biological matrixes which are used in the preparation of most biological samples, is commonly used for analysis of biological samples. Adams et al, Matrix Assisted Laser Desorption Ionization Time of Flight Mass Spectrometry Analysis of Zeins in Mature Maize Kernels, J. Agric. Food Chem., 52: 1842-49 (2004) discuss analysis and identification of zeins from crude maize kernel prolamin extracts by MALDI-TOF MS.

Dombrink-Kurtzman, Examination of Opaque Mutants of Maize by Reversed-Phase High-Performance Liquid Chromatography and Scanning Electron Microscopy, Journal of Cereal Science, 19: 57-64 (1994) discusses examination of zein proteins of eight opaque maize mutants by RP-HPLC and examination of the micro-structure of their endosperms by scanning electron microscopy (SEM). (Based on results of the study using RP-HPLC and SEM, the author proposes that zeins are not responsible for hardness of kernels.)

Dien et al, Fate of Bt Protein and Influence of Corn Hybrid on Ethanol Production, Cereal Chemistry, 79(4): 582-585 (2002) discuss the presence of Bt protein in corn co-products at various stages during production of fuel ethanol. After comparing ethanol yield from five corn hybrids, the authors propose that the chemical structure of starch and the starch-protein matrix may affect starch availability.

Philippeau et al, Influence of Grain Source on Ruminal Characteristics and Rate, Site, and Extent of Digestion in Beef Steers, Journal of Animal Science, 77:1587-1596 (1999) discuss inverse correlation between microbial protein synthesis and rumen starch degradation. The authors propose that the site and extent of starch degradation depends on the nature of the cereal (for example, wheat versus corn) and the genotype of the cereal.

Zinn et al, Flaking corn: Processing Mechanics, Quality Standards, and Impacts on Energy Availability and Performance of Feedlot Cattle, Journal of Animal Science, 80:1145-1156 (2002) discuss how the extent of starch digestion can be increased by flaking corn. The authors propose that this increase is caused by disrupting the protective protein matrix around the starch granule.

### SUMMARY

The present disclosure provides a method for screening to predict a trait of interest from at least one plant comprising measuring the degree of starch-protein association in the plant. In some embodiments, the trait of interest is fermentability to yield ethanol from at least one plant. In other embodiments, the trait of interest is digestibility. The method comprises measuring the degree of starch-protein association in the plant. The degree of starch-protein association can be determined by identifying either chemical properties or physical properties, or both properties of plant cells.

In one embodiment, there is provided a method of measuring the degree of starch-protein association that comprises measuring a chemical property of the plant determined by analysis of protein, starch or both.

In another embodiment, there is provided a method of measuring the degree of starch-protein association that comprises measuring a physical property of the plant determined by visualization of protein, starch, or both.

The present disclosure also provides a method for predicting fermentability to yield ethanol from at least one plant comprising measuring the degree of starch-protein association in the plant, wherein starch-protein association is determined by a combination of analyzing plant protein and visualizing protein packing within starch-protein association.

The present disclosure also provides a method for predicting digestibility from at least one plant comprising measuring the degree of starch-protein association in the plant, wherein starch-protein association is determined by a combination of analyzing plant protein and visualizing protein packing within starch-protein association.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is an overlay of mass spectra analysis of total zein proteins from corn samples diluted 5-fold with matrix solution. High-ethanol yield and low-ethanol yield hybrids can be distinguished by peak height, with low-ethanol yield hybrids showing higher peaks at each of the indicated zein protein markers.

FIG. 2 is an overlay of RP-HPLC chromatograms profiling zein proteins in high-ethanol yield and low-ethanol yield hybrids. The low-ethanol yield hybrid demonstrates larger peak areas at 66.7 minutes than does the high-ethanol yield hybrid.

FIG. 3 shows sections of corn endosperm (A and B) and suspensions of starch grains (C and D) observed under polarized light microscopy, following hands-free sectioning of corn endosperm tissue from kernels of high-ethanol yield and low-ethanol yield hybrids.

FIG. 4 shows confocal images of hand-free endosperm cross-sections stained for starch (black) and protein (fluorescence, gray), from corn kernels of high-ethanol yield (A) and low-ethanol yield (B) hybrids. Note the differences between the patterns of organization of the protein matrix (gray) and starch grains (black) within the cells.

FIG. 5 shows tri-dimensional projections of the protein matrix of endosperm cells from cross-sections of corn kernels of high-ethanol and low-ethanol yield hybrids, obtained from sequence series of confocal optical sections of samples stained for protein.

FIG. 6 shows cryo-scanning electron micrographs of hand-free cross sections of corn endosperm from high-ethanol (A to C) and low-ethanol (D to F) yield hybrids. Note differences in amyloplast packing, and the presence of material(s) attached to each amyloplast, which staining and observation by fluorescence and confocal microscopy revealed to be mainly protein, rich in thiols and disulfides.

FIG. 7 shows a transmission electron micrograph of corn endosperm cells from a sample of a high-ethanol yield hybrid (EA).

FIG. 8 shows a transmission electron micrograph of corn endosperm cells from a sample of a low-ethanol yield hybrid (EJ).

FIG. 9 shows a transmission electron micrograph of amyloplasts in a corn endosperm cell from a sample of a high-ethanol yield hybrid (5494), fixed by high-pressure freezing.

FIG. 10 shows transmission electron micrographs of amyloplasts in corn endosperm cells from samples of a low-ethanol yield hybrid (5110), fixed by high-pressure freezing.

FIG. 11 is a chart showing percent of starch grains associated with protein counted from thin sections from high-ethanol (EA) and low-ethanol (EJ) yield hybrids.

FIG. 12 shows thin sections of corn kernels stained for protein (fluorescence, gray).

### DETAILED DESCRIPTION

The following description is merely exemplary in nature and is not intended to limit the present disclosure, application, or uses.

In accordance with the present disclosure, Applicants have discovered that the relative level of digestibility and/or fermentability to yield ethanol of an individual plant variety can be predicted by measuring the degree of starch-protein association in the plant. A characteristic, highly organized, protein matrix consisting of numerous, tightly packed protein bodies, pressed against amyloplasts, is present in the endosperm cells of low-ethanol yield and low digestibility plants. Plants with such characteristics have cells that are more difficult to break apart and release cell contents, as single, protein-free starch grains. While not bound by theory, it is believed that the ability to resist breaking apart, or a greater degree of starch-protein association, may be a major limitation on digestibility and the economic production of ethanol from plant sources since the availability of starch grains is reduced. In the process of fermentation and digestion, starch grains are broken down to simple sugars, typically by the addition of alpha amylase and/or gluco amylase. Ethanol is produced when yeast feed on the sugars.

As used herein, the phrase “degree of starch-protein association” indicates the level to which starch and protein are connected to each other as determined by, for example, the methods described below. An example of a starch-protein association includes but is not limited to amyloplasts in association with protein bodies.

Screening hybrids from a mixture for a trait of interest typically precedes processing of the grain by milling, cooking, etc., and can start with a study of subcellular organization of endosperm cells, for example, a study of the subcellular organization of endosperm cells of high-ethanol and low-ethanol yield hybrids or of high and low digestibility hybrids. High-ethanol and low-ethanol yield varieties have distinguishable characteristics both in chemical and physical properties as do high and low digestibility hybrids, and identification of these characteristics leads to predicting and screening a plant for the trait of interest.

The inventors have discovered that plants' chemical properties, assessed using chromatographic analyses, show distinctly different protein elution profiles for high and low fermentable plant lines. In particular, for example, specific plant proteins such as zeins are highly more abundant in low fermentable corn lines in comparison with high fermentable corn lines. Zein proteins are hydrophobic and are found bound to starch through non-covalent bonding and hydrophobic interactions. Accordingly, higher zein content can play an important role in the fermentation yield process such as inhibiting the fermentation process by limiting the starch availability. Zein proteins contain higher amounts of thiols and disulfides relative to other proteins, thus, in one embodiment, quantification of thiols and disulfides in a protein sample is an indicator of the amount of zein protein.

Similarly, plants' chemical properties show distinctly different protein elution profiles for high and low digestibility plant lines. Zein proteins are more abundant in low digestibility corn lines and less abundant in high digestibility corn lines.

The inventors have further determined that plants' physical properties, assessed using microtechniques, reveal that each of high-ethanol and low-ethanol yield plants has distinguishable subcellular organizations as do high and low digestibility plants. No significant differences are found between starch grains of high-ethanol and low-ethanol yield hybrids in terms of size, shape, indices of refraction, ratios of starch grain populations, and color of staining. However, in samples of high-ethanol yield hybrids, starch grains are randomly dispersed inside the cell, easy to isolate, thus forming suspensions containing higher densities of starch grains. In such high-ethanol yield samples, starch grains are generally dispersed in suspension as single structures, rarely associated with protein, whereas, for samples of low-ethanol yield hybrids, starch grains are highly organized inside the cell, difficult to isolate, thus resulting in low-density-starch grain suspensions. These low-density-starch grains are frequently present in suspension as aggregates or clusters, and are frequently associated with protein. Specifically, microscopic examination shows that the starch grains of high-ethanol yield hybrids are loosely packed inside the cells and rarely show irregular surfaces. Starch grains of low-ethanol yield hybrids are tightly packed against each other, and show materials associated with/or on the amyloplast surface. These same findings apply to other traits of interest including digestibility.

Protein staining shows significant differences between high-ethanol and low-ethanol yield hybrids: the protein matrix of high-ethanol yield samples is smooth, continuous, and fragile, but the protein matrix of low-ethanol yield samples is irregular, thicker, with a high density of globular structures. Therefore, the grains dispersed in aggregates or clusters and associated with proteins can be evaluated as low-ethanol yield variety. The findings are similar for high and low digestibility hybrids. The phrase “protein packing” as used herein describes the visualization of the protein matrix. In some embodiments, visualization of protein packing is used to analyze starch-protein association.

The phrase “starch protein matrix” as used herein refers to the association of starch with surrounding protein matrices, usually in endosperm cells.

In accordance with the inventors' findings above, it is possible to predict traits such as fermentability to yield ethanol or digestibility by a method of analyzing the plant hybrid properties. Use of such a method to predict fermentability to yield ethanol can lead to selection of preferred grain properties for optimum process conditions in the fermentation of grains or biomass. Use of such a method to predict digestibility can lead to selection of preferred grain properties for optimum feed design. To select a plant variety preferable for a particular trait, a method of the present invention involves the generation of chemical, kinetic, physical, rheological, morphological, and agronomic information for a representative population with a wide range of variation. Such information may be generated using the application of a destructive or non-destructive technique or a combination thereof. In some embodiments, the invention employs a technique to analyze at least one chemical or physical property or both.

Higher concentration of a certain substance can reveal information regarding a trait of interest, for example, fermentability of a plant to yield ethanol. Thus, analyzing chemical properties of a plant can be carried out through profiling a certain substance of cells or tissues taken from the plant. A wide variety of substances can be evaluated for the purpose of screening plants and plant varieties. Generally, a substance to be analyzed will be selected based upon species of the plant to be analyzed. At least one substance needs to be analyzed and an ordinarily skilled artisan can determine the optimal or preferable number of target substances based on the plant to be used. Typically, a substance to be analyzed is selected from the group consisting of proteins, starches, and lipids.

Any chemical analysis techniques known in the art can be used for the determination of chemical properties, such as determination of protein, starch, and lipid compositions. Among various chemical analysis techniques, separation techniques are generally desirable for an application of the present invention. Examples of chemical analysis techniques include, but are not limited to, HPLC, MALDI-TOF MS, capillary electrophoresis, RP-HPLC on-line MS, gel electrophoresis, and combinations thereof.

In one embodiment, a method of predicting a trait of interest is a high-throughput method employing a high-throughput analyzer capable of producing results quickly. Fast delivery of the result on a trait such as fermentability can help in optimizing a fermentation process at a plant level. Illustrative analyzers include but are not limited to, for example, capillary electrophoresis, RP-HPLC on-line MS, gel electrophoresis, and combinations thereof.

The term plant as used herein refers to an individual plant, more than one plant, a plant variety, a crop breed, or a crop variety. A plant to be analyzed by the methods herein can be any plant that is fermentable through conventional ethanol production methods. Typically, the plant is a cereal variety such as, for example, maize, wheat, barley, rice, rye, oat, sorghum, or soybean. In some embodiments, the plant is analyzed for the chemical profile of target substances such as protein, starch, or lipid. In a particular embodiment, the plant is analyzed for at least one zein protein which comprises α-zein, β-zein, and γ-zein proteins. In other embodiments, the sample is analyzed to determine sulfur content, an indicator of thiol and disulfide containing proteins.

Analysis of a plant can include analysis of one or more seeds from the plant. Any seed can be utilized in a method or assay of the invention. Individual seeds or a plurality of seeds can be analyzed.

Analysis of a plant can include analysis of other plant tissues. As used herein, plant tissues include but are not limited to, any plant part such as leaf, flower, root, and petal.

A trait of interest can also be predicted by analyzing physical properties of the plant, for example starch-protein association. In one embodiment, the method comprises determining the starch density of a sample of the plant in suspension. Starch density is the amount of starch visualized or measured in some discrete unit, for example, a volume or an area of an image. In another embodiment, the method comprises analyzing protein through immunoprecipitation or immunostaining. In another embodiment, the method comprises taking a tissue sample from at least one plant; staining the tissue sample with a stain reagent for protein, lipid, lipoprotein, or carbohydrate; observing or obtaining images of the stained sample with a microscope or equivalent equipment; and determining starch-protein association by observing or analyzing the images.

Visualization of cell components generally requires sample preparation as an initial step. Samples for microscopic analysis can be taken from any part or tissue of the plant of interest. Generally, it is desirable to obtain samples from plant parts or tissues which are a major starch source. Illustratively, endosperm tissues can be used for sample preparation. More than one sample can be taken from one plant variety to confirm the accuracy of the analysis. The samples can be either sectioned (thin, flat slices) or grind (scratched with a razor blade or ground in a mechanical grinder to form powder). If two or more plants are analyzed, samples from each plant should generally be obtained from the same tissue.

After samples are taken from the plants, they can be stained or labeled for better microscopic observation. Staining targets can be changed depending upon the plant to be used in production. The targets are generally selected from protein, lipid, lipoprotein, and carbohydrate. Staining procedures are well known in the art and practically any known procedure can be successfully employed for the present invention. A specific staining procedure will be suitably selected in accordance with the staining target. Like staining protocols, any known staining reagent can be used for the present invention. Illustratively, mercurochrome, iodine, and Sudan IV can be used for protein, starch, and lipid staining, respectively. However, the choice of reagents is not necessarily determinative for the outcome of the invention. Samples can be stained with one or more reagents. For example, a sample can be stained with mercurochrome to identify proteins containing thiols and disulfides, then counterstained with acridine orange to identify amyloplasts. Double-staining in this manner allows visualization of co-localized targets.

To analyze physical properties of the plant, microscopy imaging techniques can be employed. Any known microscopy imaging technique such as, for example, light, confocal, and electron microscopy, can be used to determine subcellular organization of cells or tissues. An ordinarily skilled artisan can choose suitable imaging techniques for use in accordance with the method of the invention. For example, suitable imaging techniques may include, but are not limited to, differential interference contrast (DIC) microscopy, polarized light microscopy, fluorescence microscopy, epi-fluorescence microscopy, confocal microscopy, scanning electron microscopy (SEM), transmission electron microscopy (TEM), and hyperspectral imaging.

The samples are imaged to identify subcellular organization within the samples. For example, the respective amounts of starch grains associated with protein and without protein present in the plant samples can be determined by counting of associated grains. This can serve as the basis for determining high-ethanol and low-ethanol yield traits or for determining high and low digestibility traits. Observation and counting can be conducted by direct observation through an eyepiece and/or examination of images obtained by the imaging techniques described above. Starch-protein association can be determined by quantification of fluorescent dots, determination of fluorescence intensity or determination of area of fluorescence. Analysis of subcellular organization, such as counting of grains, can be automated with the assistance of a computer device or software, or combination of both computer device and software.

Other visualizing techniques can be employed to analyze a plant's physical and chemical characteristics, including but not limited to fluorescent plate reader, fluorimeter, flow cytometer, spectrophotometer, light scatter, and hyperspectral analysis.

Target plants which can be used in the physical analysis method can be any fermentable plants. Illustratively, the plants are the same as those which are listed above in the chemical analysis method.

In one embodiment, the degree of starch-protein association can also be determined by combination of chemical analysis and physical analysis of the target plant. The order of conducting the analyses does not generally influence the outcome. Any one analysis can be done first and the other analysis is used later to confirm the first result. The combination of the two analyses can, in some embodiments, provide more accurate results than single analysis.

Also provided herein is a multivariate analysis model for predicting fermentability to yield ethanol from a plant. The model comprises: (a) obtaining a sample from at least one plant; (b) measuring in the sample at least one chemical property, at least one physical property, at least one agronomic property, or any combination thereof; and (c) determining correlation between the at least one property and fermentability to yield ethanol.

The at least one chemical property can be selected from the group consisting of oil content, fiber content, moisture content, amino acid content, protein content, and starch content. Oil content can include both the amount and type of oil. Fiber content can include both the amount and classification of fiber. Amino acid content can include both the amount and type of amino acid. Protein content can include both amount and type of protein. Starch content can include both amount and classification of starch.

The at least one physical property can be selected from the group consisting of absolute seed density, seed test weight, seed hardness, seed size, hard to soft endosperm ratio, germ size, color, cracking, water uptake, pericarp thickness, and crown size.

The at least one agronomic property can be selected from the group consisting of crop yield, seed vigor, relative maturity, emergence vigor, vegetative vigor, stress tolerance, disease resistance, branching, flowering, seed set, seed density, standability, and seed handling. Relative maturity as used herein is the cessation of dry weight accumulation by the kernel and, therefore, maximum yield. Seed handling as used herein includes packing density, fragility, moisture content, threshability, etc.

The invention is further illustrated in but not limited by the following examples. Variations of the following examples are possible without departing from the scope of the invention.

### EXAMPLES

Abbreviations used in the following examples include:
ACN Acetonitrile
DTT Dithiothreitol
RP-HPLC Reverse phase high performance liquid chromatography
MALDI-TOF MS Matrix assisted laser desorption ionization time-of-flight mass spectroscopy
SEM Scanning electron microscopy
TEM Transmission electron microscopy

### Example 1

Chemical analysis using RP-HPLC and/or MALDI-TOF MS. Protein was extracted from corn samples by resuspending defatted corn flour (50 mg) in 25 mM NH₄OH, 60% ACN, and 10 mM DTT, then shaking at 60° C. (in a water bath) for two hours. Supernatant containing protein was recovered by centrifugation (3000 rpm for 10 minutes at room temperature) and transferred to empty tubes. Each sample was analyzed by MALDI-MS and RP-HPLC.

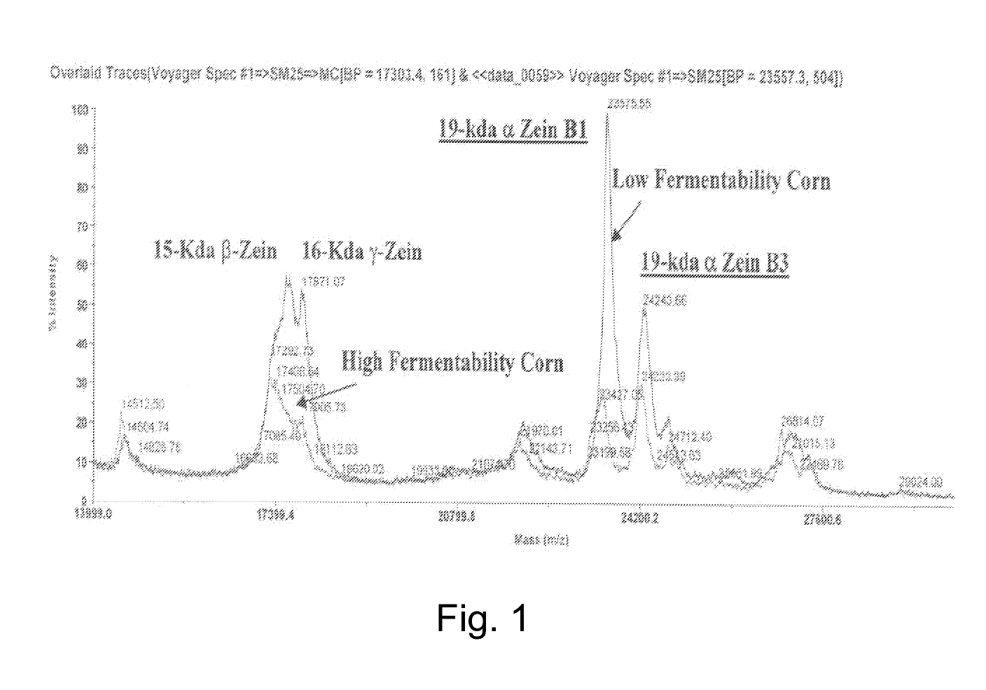

MALDI-TOF MS was performed on diluted protein samples (diluted 5 fold with JAVA matrix solution, Sigma, St. Louis, Mo.). Mass spectra were obtained using an Applied Biosystems Voyager-DE PRO Biospectrometry. FIG. 1 is an overlay of mass spectra analysis of total zein proteins from corn samples diluted 5-fold with matrix solution. High-yield and low-ethanol yield hybrids can be distinguished by peak height, with low-ethanol yield hybrids showing higher peaks at each of the indicated zein protein markers.

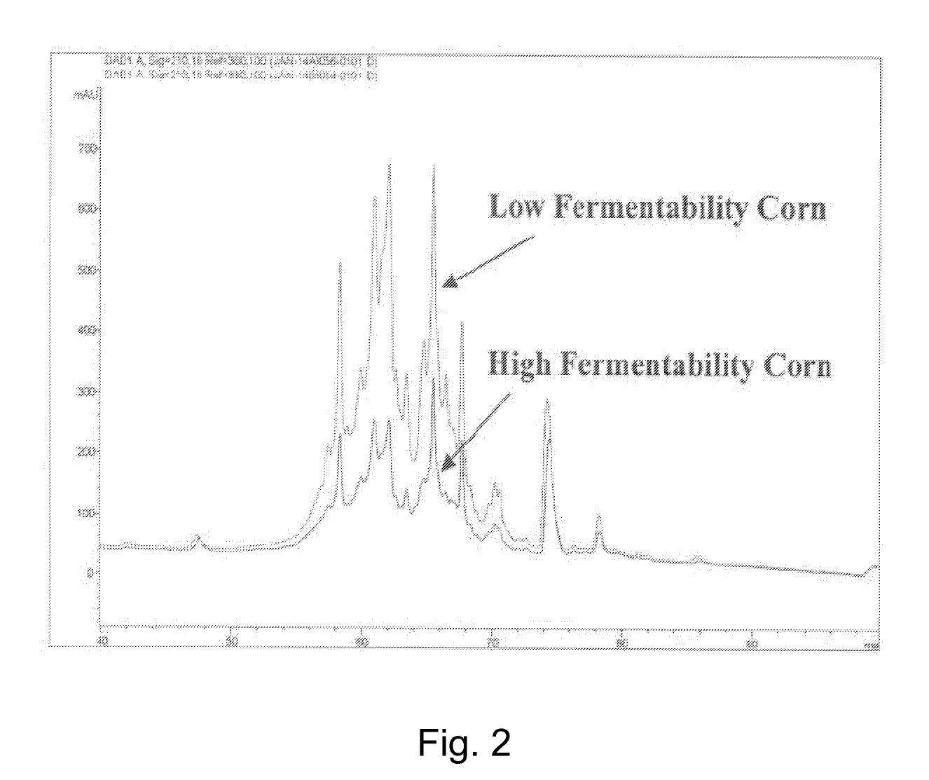

RP-HPLC was performed by injecting protein samples on a C18 Vydac HPLC column and a linear gradient of acetonitrile (from 15% to 80%). Entire samples were collected; sample fractions were collected at 67 minutes for subsequent analysis by MALDI-TOF MS. FIG. 2 is an overlay of RP-HPLC chromatograms profiling zein proteins in high-yield and low-ethanol yield hybrids. The low-yield hybrid demonstrates larger peak areas at 66.7 minutes than does the high-yield hybrid.

### Example 2

Identification of physical properties of a sample plant using microscopy techniques was carried out with two kinds of corn hybrids, i.e., hand-pollinated hybrids (Table 1) and open-pollinated hybrids (Table 2).

1. Sample Preparation for Hand-Pollinated Hybrids

A blind assay was conducted with samples consisting of 12 hybrids randomly collected from spare seed from previously analyzed samples and determined to be six high-ethanol and six low-ethanol yield hybrids. A total of 12 kernels were collected from each hybrid for the assay. Two to four kernels per hybrid were processed each time. Dry kernels (4 per hybrid) and kernels imbibed in water (8 per hybrid) were used. Longitudinal sections were tested for two dry and two imbibed kernels/hybrid. Transversal sections were tested for the remaining kernels (8 kernels per hybrid). Only endosperm tissue, either sectioned or grind (scratched with a razor blade to powder or by mechanical grinding) was used. The comparison between hybrids that led to the establishment of two distinct groups was based on cross sections.

The 12 hybrids were labeled EA to EL. Percent ethanol yields after fermentation of the hybrids are shown in Table 1.

TABLE 1
A- Hand-pollinated

| Label | Yield |
|---|---|
| EA | 15.67 |
| EB | 15.47 |
| EF | 15.66 |
| EG | 15.68 |
| EH | 15.98 |
| EI | 15.72 |
| EC | 14.38 |
| ED | 14.52 |
| EE | 14.64 |
| EK | 13.66 |
| EL | 14.52 |
| EJ | 13.86 |

At least six sections were obtained per kernel, and 6-12 endosperm portions processed for TEM, from which at least 8 slides, with 5 to 8 thin-sections each, were used for fluorescence/confocal microscopy, and 3 to 15 grids for TEM. The following microtechniques were used: differential interference contrast (DIC); polarized light; fluorescence and confocal microscopy (for stained sections); SEM and TEM. The selection of morphological and/or subcellular traits, or markers, was based on the following: (1) the same subcellular marker(s) had to be observed in all samples from kernels of the some hybrid; (2) the subcellular marker(s) had to be present only in 6 of the hybrids (or be characteristic of); (3) presence of such trait or marker had to be corroborated by all microtechniques used.

2. Visualization of Proteins, Starch, and Lipids

Protein staining: Samples were incubated for 1 hour, at room temperature, in a mercurochrome solution ([2,7-dibromo-4-(hydroxymercuri)-fluorescein disodium salt] from Sigma, St. Louis, Mo., USA) prepared in Tris buffer, pH 7.4. This stain identifies protein thiols and disulfides. Following incubation the samples were washed in Tris buffer, mounted in water, buffer, or Vectashield (Vector Laboratories, CA, USA) and observed under a fluorescent or confocal microscope. Proteins were identified as red fluorescence upon excitation of the mercurochrome (fluorescence microscope: 525/565 nm or 545/>590 nm; confocal: 533 nm).

Starch staining: Samples were incubated for 3 to 15 min (depending on type of sample) at room temperature in commercial Lugol's Iodine solution (Electron Microscopy Sciences, PA, USA). This stain identifies starch. Following incubation the samples were washed in water, mounted in buffer, and observed under a light microscope, epi-fluorescence microscope, or confocal microscope. Starch stains dark brown. This stain was frequently used following protein stain. Staining with 1 μg/mL acridine orange solution for 3 min (Molecular Probes, CA, USA) was also used in replacement of iodine stain, allowing localization of amyloplasts (starch containing organelles), which fluoresce green when excited at 510 nm.

Lipid staining: Samples were incubated for 15 min, at room temperature, in a 0.3% (w/v) Sudan IV solution prepared in 70% ethanol (Sigma, St. Louis, Mo., USA). This stain identifies total lipids. Following incubation, the samples were washed in 50% (v/v) ethanol solution, washed in water, mounted in water, and observed under a light microscope.

3. Sample Preparation for Open-Pollinated Hybrids

This assay was conducted with samples consisting of two high-ethanol and two-low ethanol yield hybrids. The respective percent ethanol yields after fermentation are illustrated in Table 2.

TABLE 2
B- Open-pollinated

| Label | Yield |
|---|---|
| 5494 | 17.47 |
| A-03 | 17.59 |
| 5110 | 16.14 |
| B-20 | 15.52 |

A total of 12 kernels were collected from each hybrid for the assay. The assay design was identical to the one followed for hand-pollinated hybrids, but only 2 to 4 kernels per hybrid were analyzed. Besides the techniques previously tested, samples 5494 and 5110 were processed for TEM using the technique of high-pressure freezing instead of chemical fixation.

4. Quantifying Starch-Protein Association for Thin Sections

Kernels from samples EA and EJ were processed for transmission electron microscopy using an optimized microwave procedure. Thin sections (0.5 μm thickness) from the same blocks as those used previously were stained for visualization of proteins using fluorescence microscopy. Starch grains with and without protein were automatically counted from six different kernel sections. An additional count was performed for sections of the same kernel, for both EA and EJ) to check for variations within kernel. Image Pro-Plus software was used to count dark spots (starch) versus small red spots/dots (protein).

5. Quantifying Starch-Protein Association for Grind Samples

This assay consisted of two high-ethanol and two low-ethanol yield hybrids, 15 mg grind kernels per hybrid, per replicate. Each 15 mg sample was stained for protein visualization, with or without amyloplast counterstaining, washed, and 20 μL aliquots (30 μg/μL) taken for observation under fluorescence/confocal microscope. Ten images were acquired per 20 μL aliquot, and the number and area of red fluorescent spots (protein) determined using Image Pro-Plus software.

6. Result

No significant differences were found between starch grains of high-ethanol and low-ethanol yield hybrids in terms of size, shape, indices of refraction, ratios of starch grain populations, and color of staining.

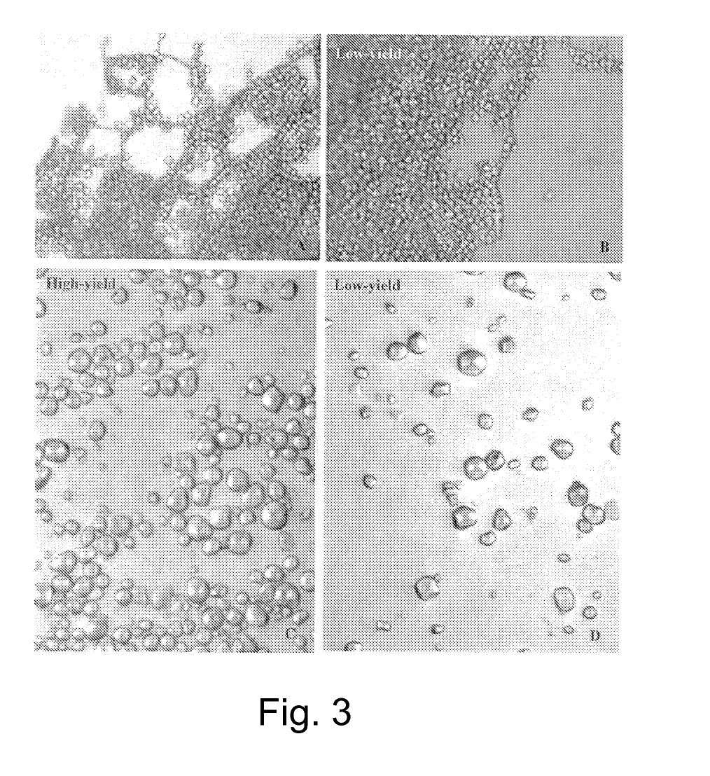

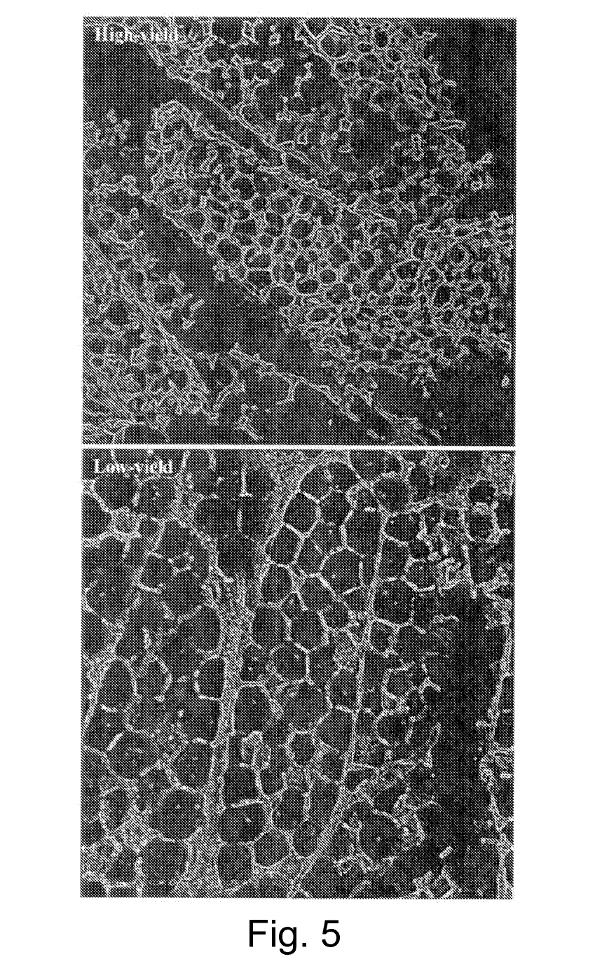

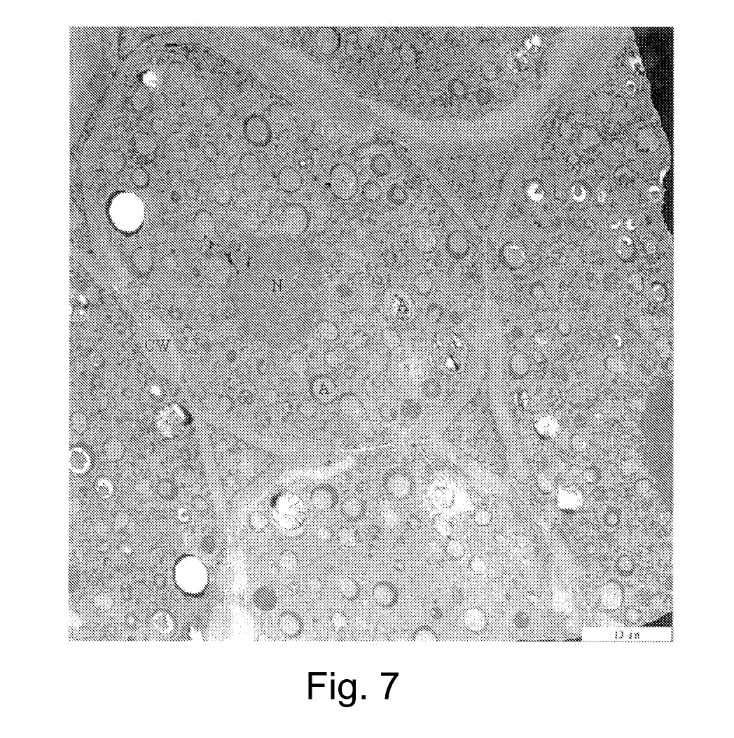

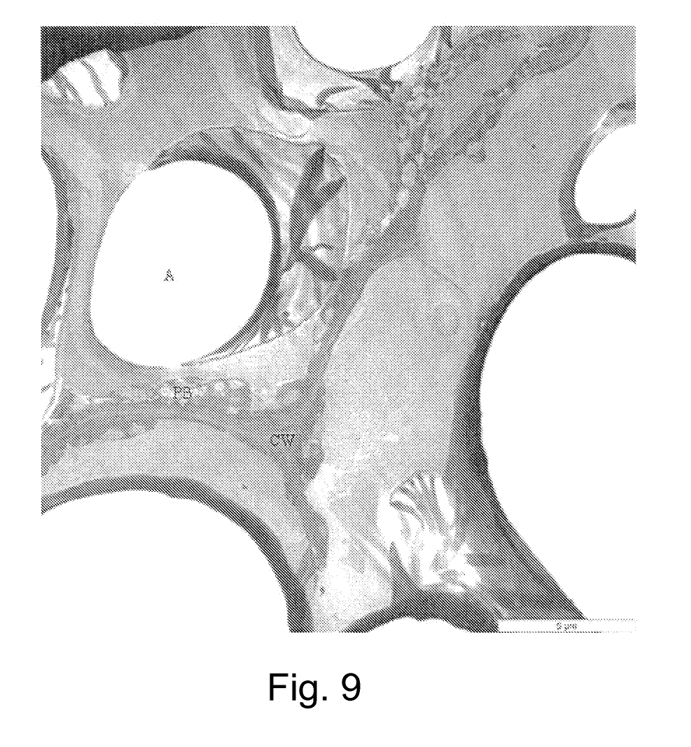

For samples of high-ethanol yield hybrids, starch grains were randomly dispersed inside the cell, easy to isolate, thus forming suspensions containing higher densities of starch grains, and such starch grains generally dispersed in suspension as single structures, rarely associated with protein. See FIGS. 3-7 and 9.

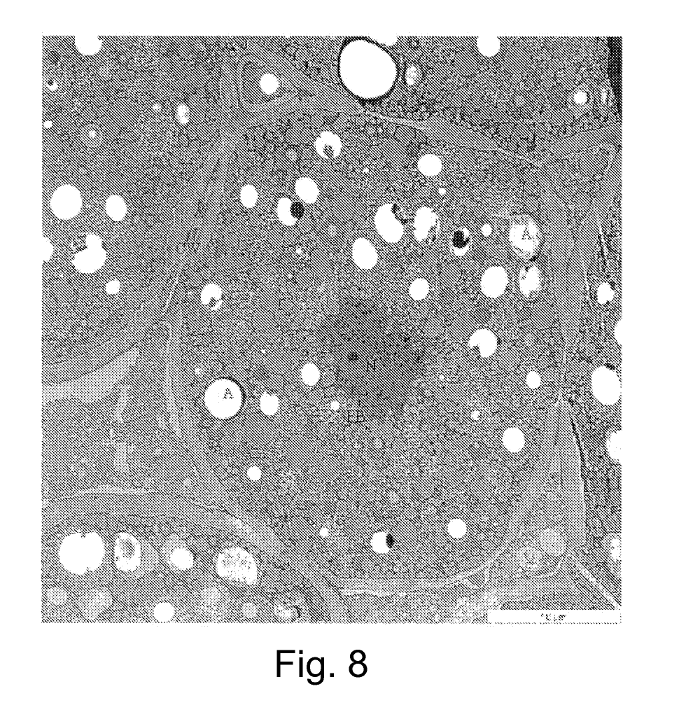

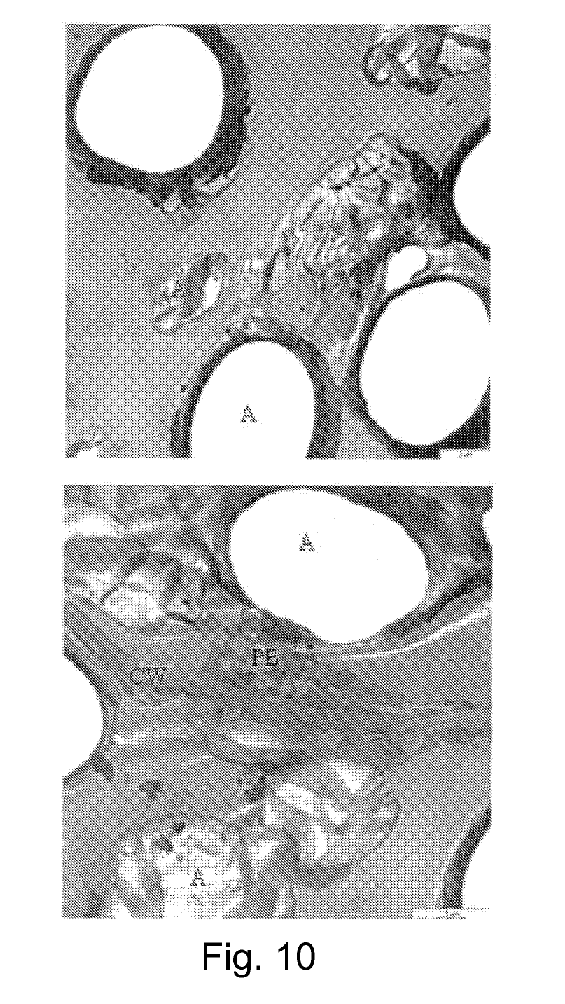

For samples of low-ethanol yield hybrids, starch grains were highly organized inside the cell, difficult to isolate, thus resulting in low-density-starch grain suspensions, frequently present in suspension as aggregates or clusters, and frequently associated with protein. See FIGS. 3-6, 8 and 10.

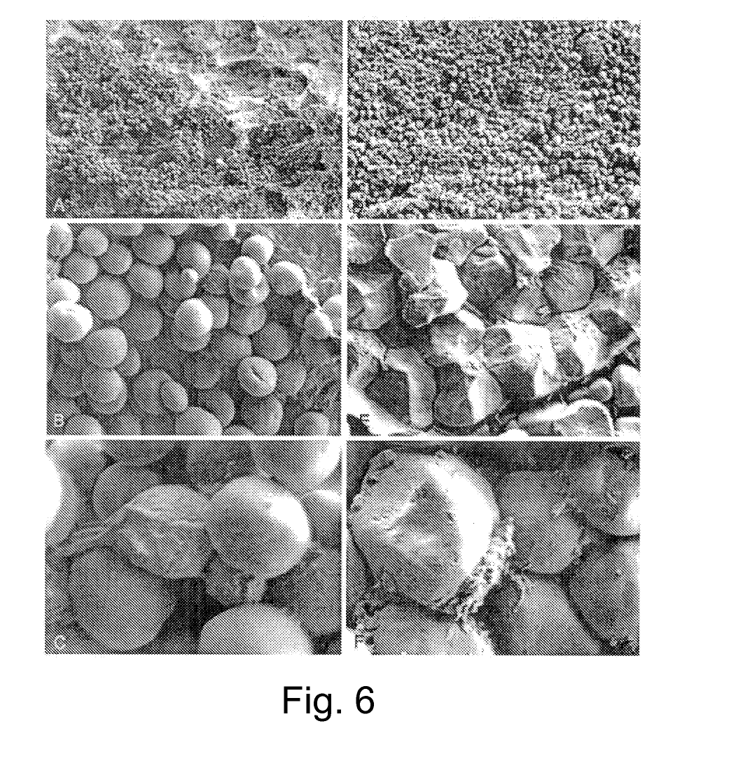

SEM results (FIG. 6) corroborated light microscopy results (FIG. 3) and showed that for high-ethanol yield hybrids the starch grains were loosely packed inside the cells, rarely showing irregular surfaces, whereas for low-ethanol yield hybrids, the grains were tightly packed against each other, showing materials associated with/or on the amyloplast surface.

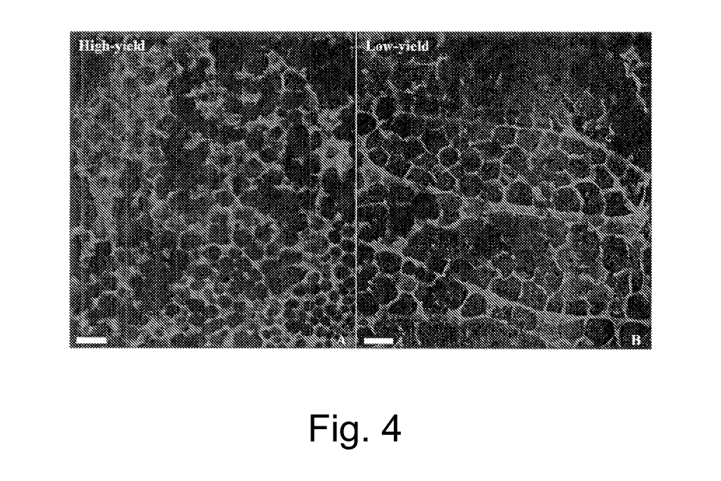

Protein staining showed considerable differences between high-ethanol and low-ethanol yield hybrids. For high-ethanol yield samples, the protein matrix was smooth, continuous, and fragile, whereas for low-ethanol yield samples, the protein matrix was irregular and thicker, having a high density of globular structures. See FIG. 4.

It was observed that starch grains from sectioned or grind endosperm of low-ethanol yield hybrids tended to disperse in suspension in lower numbers than starch grains from high-ethanol yield samples, frequently in aggregates or clusters, and were associated with proteins. Such starch-protein association was surprisingly correlated with low-ethanol yield hybrids having reduced fermentability, and thus reduced ethanol production.

Thin sections of corn endosperm samples, stained for protein, and analyzed using Image Pro-Plus software, were tested as tools to quantify starch-protein associations in low- and high-ethanol yield hybrids. Table 3 shows number of starch grains counted by Image Pro-Plus software for thin endosperm sections of corn from high- (EA) and low- (EJ) ethanol yield hybrids, stained for protein and starch, and observed under fluorescence microscopy. In the table, “clean starch” means starch grains not associated with protein and “w/protein” denotes starch grains associated with protein.

TABLE 3

| | High-ethanol yield hybrids | | | Low-ethanol yield hybrids | | |
|---|---|---|---|---|---|---|
| | Clean | w/ | | Clean | w/ | |
| | starch | protein | Total | starch | protein | Total |
| * | 1985 | 15 | 2000 | 5780 | 322 | 6102 |
| * | 2503 | 19 | 2522 | 4821 | 232 | 5053 |
| | 5027 | 54 | 5081 | 2509 | 306 | 2815 |
| | 3684 | 3 | 3687 | 2112 | 278 | 2390 |
| | 11984 | 0 | 11984 | 1419 | 270 | 1689 |
| | 7426 | 43 | 7469 | 1089 | 347 | 1436 |
| | 2937 | 4 | 2941 | 495 | 173 | 668 |
| TOTAL | 35546 | 138 | 35684 | 18225 | 1928 | 20153 |

* counts for sections from the same kernel

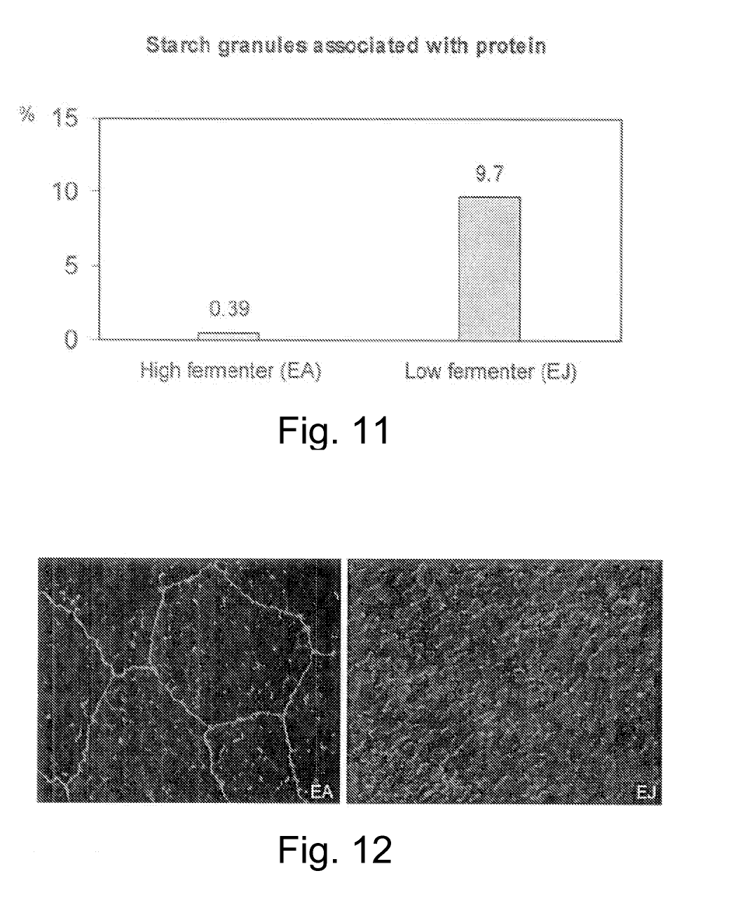

The results from Image Pro-Plus counts showed that the number of starch grains associated with protein was up to 25-fold higher in thin sections of low-ethanol yield hybrids than for high-ethanol yield ones. The comparison of the two hybrids is also illustrated in FIG. 11.

When thin corn sections were stained for protein, cell walls from the high-yield sample were brightly stained, whereas staining for cell walls from the low-yield sample was null or dim. This observation indicates that cell wall composition is different in high-ethanol and low-ethanol yield hybrids. See FIG. 12.

When introducing elements or features and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

The description of the disclosure is merely exemplary in nature and, thus, variations that do not depart from the gist of the disclosure are intended to be within the scope of the disclosure. Such variations are not to be regarded as a departure from the spirit and scope of the disclosure.

What is claimed is:

1. A method for screening at least one plant to predict a trait of interest, the method comprising measuring the degree of starch-protein association in the plant.

2. The method of claim 1, wherein the trait of interest is fermentability to yield ethanol.

3. The method of claim 1, wherein the trait of interest is digestibility.

4. The method of claim 1, wherein measuring the degree of starch-protein association comprises measuring a chemical property of the at least one plant determined by analysis of protein, starch or both protein and starch.

5. The method of claim 4, wherein the at least one plant is selected from the group consisting of maize, wheat, barley, rice, rye, oat, sorghum and soybean.

6. The method of claim 5, wherein the at least one plant is maize and the protein to be analyzed comprises at least one zein protein.

7. The method of claim 6, wherein the at least one zein protein is selected from one or more of α, β, and γ-zeins.

8. The method of claim 4, wherein measuring the degree of starch-protein association comprises analyzing protein to determine sulfur content.

9. The method of claim 4, wherein measuring the degree of starch-protein association comprises analyzing protein by a separation technique selected from the group consisting of HPLC, MALDI-TOF MS, capillary electrophoresis, RP-HPLC on-line MS, gel electrophoresis, SDS page, 2-dimensional gel electrophoresis, and combinations thereof.

10. The method of claim 9, wherein the separation technique is HPLC, MALDI-TOF MS or both HPLC and MALDI-TOF MS.

11. The method of claim 1, wherein measuring the degree of starch-protein association comprises measuring a physical property of the plant determined by visualization of protein, starch or both protein and starch.

12. The method of claim 11, wherein measuring the starch-protein association comprises obtaining a plant tissue sample and determining starch density in a suspension of the sample.

13. The method of claim 11, wherein measuring the starch-protein association comprises obtaining a plant tissue sample and analyzing protein by immunostaining or immunoprecipitation.

14. The method of claim 11, wherein measuring the starch-protein association comprises:
(a) taking a tissue sample from the at least one plant;
(b) staining the tissue sample with a stain reagent for protein, lipid, lipoprotein, and/or carbohydrate;
(c) observing or imaging the stained sample under a microscope; and
(d) measuring starch-protein association.

15. The method of claim 14, wherein the at least one plant is selected from the group consisting of maize, wheat, barley, rice, rye, oat, sorghum and soybean.

16. The method of claim 14, wherein the tissue sample is taken from endosperm.

17. The method of claim 14, wherein the stain reagent is mercurochrome, Sudan IV or iodine.

18. The method of claim 14, wherein the microscope is selected from the group consisting of differential interference contrast (DIC) microscope, polarized light microscope, fluorescence microscope, epi-fluorescence microscope, confocal microscope, scanning electron microscope (SEM), hyperspectral microscope, and transmission electron microscope (TEM).

19. The method of claim 14, wherein determining starch-protein association is made by quantification of fluorescent dots, determination of fluorescence, fluorescence intensity, or determination of area of fluorescence.

20. A method for predicting fermentability to yield ethanol from at least one plant comprising measuring the degree of starch-protein association in the plant, wherein starch-protein association is determined by analyzing plant protein and visualizing protein packing within starch-protein association.

21. The method of claim 20, wherein measuring the starch-protein association comprises analyzing the protein and confirming the results of analysis of the protein by visualization of the protein packing.

22. The method of claim 20, wherein measuring the starch-protein association comprises analyzing plant protein by staining the plant protein with mercurochrome and visualizing protein packing.

23. A method for predicting digestibility from at least one plant comprising measuring the degree of starch-protein association in the plant, wherein starch-protein association is determined by analyzing plant protein and visualizing protein packing within starch-protein association.

24. The method of claim 23, wherein measuring the starch-protein association comprises analyzing the protein and confirming the results of analysis of the protein by visualization of the protein packing.

25. The method of claim 23, wherein measuring the starch-protein association comprises analyzing plant protein by staining the plant protein with mercurochrome and visualizing protein packing.

26. An assay for screening at least one plant to predict a trait of interest, the assay comprising:
(a) obtaining a sample from the plant, and
(b) measuring in the sample starch-protein association, wherein the degree of starch-protein predicts the trait of interest.

27. The assay of claim 26, wherein the trait of interest is fermentability to yield ethanol.

28. The assay of claim 26, wherein the trait of interest is digestibility.
